(19) United States
(12) Patent Application Publication
(10) Pub. No.: US 2025/0057649 A1
(43) Pub. Date: Feb. 20, 2025

(54) Prosthetic Atrioventricular Valve with Large Waist

(71) Applicant: St. Jude Medical, Cardiology Division, Inc., St. Paul, MN (US)

(72) Inventors: William H. Peckels, Robbinsdale, MN (US); Preston James Huddleston, Maplewood, MN (US); Heath Marnach, Minneapolis, MN (US); Son Mai, North Branch, MN (US)

(73) Assignee: St. Jude Medical, Cardiology Division, Inc., St. Paul, MN (US)

(21) Appl. No.: 18/778,173

(22) Filed: Jul. 19, 2024

Related U.S. Application Data
(60) Provisional application No. 63/519,576, filed on Aug. 15, 2023.

(51) Int. Cl. A61F 2/24

(52) U.S. Cl. CPC A61F 2/2418; A61F 2220/0016; A61F 2220/0025; A61F 2250/0039; A61F 2250/0069

(57) ABSTRACT

A prosthetic heart valve may include a collapsible and expandable frame that includes an atrial anchor, a ventricular anchor, and a center waist extending therebetween. The frame may include a plurality of commissure attachment features (“CAFs”) that include struts that extend from the center waist. A plurality of prosthetic leaflets may be mounted to the plurality of CAFs. A sealing fabric may be coupled to an outer surface of the frame. When the valve is expanded, the atrial and ventricular anchors each flare outwardly from the center waist, which defines a waist diameter of the frame. When expanded, each of the CAFs may be spaced from each other so that gaps in the frame are present therebetween. When expanded, the CAFs may be positioned along an imaginary circle having a diameter that is smaller than the waist diameter.

### CROSS-REFERENCE TO RELATED APPLICATIONS

This application claims priority to the filing date of U.S. Provisional Patent Application No. 63/519,576, filed Aug. 15, 2023, the disclosure of which is hereby incorporated by reference herein.

### BACKGROUND OF THE DISCLOSURE

Heart valve disease is a significant cause of morbidity and mortality. One treatment for this disease is valve replacement. One form of replacement device is a bioprosthetic valve. Collapsing these valves to a smaller size or into a delivery system enables less invasive delivery approaches compared to conventional open-chest, open-heart surgery. Collapsing the implant to a smaller size and using a smaller delivery system minimizes the access site size and reduces the number of potential periprocedural complications.

The size to which an implant can be collapsed is limited by the volume of materials used in the implant, the strengths and shapes of those materials, and the need to function after expansion (or re-expansion). Using multiple steps and/or multiple delivery system devices may increase the time and complexity of a procedure.

Native atrioventricular valves (i.e., the tricuspid valve and the mitral valve) typically have a larger size and/or diameter compared to the native aortic valve and the native pulmonary valve. Among the native atrioventricular valves, a regurgitant tricuspid valve typically has a larger size and/or diameter than a regurgitant mitral valve. For example, for patients with severe tricuspid valve regurgitation, the diameter of the tricuspid valve may range from about 40 mm to about 65 mm, although these numbers are merely exemplary. As a result, prosthetic heart valve designs and considerations for replacing the different native heart valves are not identical. For example, to accommodate the large size of the mitral and tricuspid valve, recent prosthetic heart valve designs have included an outer frame with a large size to engage the native mitral or tricuspid annulus, and a smaller and generally cylindrical inner frame within that outer frame, the inner frame housing the prosthetic valve leaflets. However, this double-stented design generally increases the bulk of the prosthetic heart valve, resulting in a larger profile when collapsed within a delivery device. This, in turn, requires the delivery device (e.g., a catheter housing the collapsed prosthetic heart valve for delivery) to have a larger size to accommodate the large prosthetic heart valve. Typically, it is desirable for catheters of transcatheter heart valve delivery devices to have a smaller size, since the catheters may need to pass through the vasculature to reach the native heart valve in a minimally invasive manner. Thus, it would be desirable to have a prosthetic heart valve that is able to fit within a native tricuspid or mitral valve but is able to collapse to a small size to be accommodated in a relatively small profile delivery device.

### BRIEF SUMMARY OF THE DISCLOSURE

According to one aspect of the disclosure, a prosthetic heart valve for replacing a native atrioventricular valve includes a collapsible and expandable frame. The frame may include an atrial anchor, a ventricular anchor, and a center waist extending between the atrial anchor and the ventricular anchor. The frame may include a plurality of commissure attachment features (“CAFs”) that include struts that extend from the center waist. A plurality of prosthetic leaflets may be mounted to the plurality of CAFs. A scaling fabric may be coupled to an outer surface of the frame. In an expanded condition of the prosthetic heart valve, the atrial anchor and the ventricular anchor each flare outwardly from the center waist, the center waist defining a waist diameter of the frame. In the expanded condition of the prosthetic heart valve, each of the plurality of CAFs may be spaced from adjacent ones of the plurality of CAFs so that gaps in the frame are present between adjacent ones of the plurality of CAFs. In the expanded condition of the prosthetic heart valve, the CAFs may be positioned along an imaginary circle having a diameter that is smaller than the waist diameter.

In the expanded condition of the prosthetic heart valve, the waist diameter may be about 40 mm. In a collapsed condition of the prosthetic heart valve, the prosthetic heart valve may have an axial height between an inflow end of the prosthetic heart valve and an outflow end of the prosthetic heart valve, and the axial height may be between about 48 mm and about 58 mm. A commissure support ring may be coupled to and may extend around the plurality of CAFs. The commissure support ring may be a collapsible and expandable structure that has a circular or lobed shape in an expanded condition of the commissure support ring. The commissure support ring may include a first circumferential row of generally diamond-shaped cells. The commissure support ring may include a second circumferential row of generally diamond-shaped cells adjacent the first circumferential row. The commissure support ring may include a plurality of connectors integrally formed with the commissure support ring, the plurality of connectors each having a shape that is complementary to a shape of each of the plurality of CAFs.

The frame may include a plurality of tines on the ventricular anchor, and each of the plurality of tines may extend to a free end pointing toward the atrial anchor in a collapsed condition of the prosthetic heart valve. In the expanded condition of the prosthetic heart valve, the ventricular anchor of the frame may be bell-shaped. The sealing fabric may extend over the ventricular anchor and over the center waist of the frame, and an inflow edge of the sealing fabric may be positioned a spaced distance from a terminal end of the atrial anchor. In the expanded condition of the prosthetic heart valve, the struts that couple the plurality of CAFs to the center waist may include a first contour extending in an outflow and radially inward direction, and a second contour extending in an outflow and radially outward direction, the plurality of CAFs being coupled to the second contour of respective ones of the struts. In the expanded condition of the prosthetic heart valve, the plurality of CAFs may each extend in a direction substantially parallel to a center longitudinal axis of the prosthetic heart valve. In the expanded condition of the prosthetic heart valve, the diameter of the imaginary circle may be about 29 mm.

Each of the plurality of prosthetic leaflets may include an attached edge, a free edge opposite the attached edge, and two side edges extending between the free edge and the attached edge. An atrial skirt may be attached to an interior surface of the frame. The atrial skirt may have an inflow edge attached to the atrial anchor, and a plurality of contoured sections, each of the plurality of contoured sections attached to a respective one of the attached edge of the plurality of prosthetic leaflets. The atrial skirt may be formed of a material that is non-permeable to blood. The material may be a woven polyester. The atrial skirt may be coupled to the sealing skirt. The atrial skirt may include a plurality of extensions. Each of the plurality of extensions may be positioned between a respective one of the plurality of CAFs and a respective commissure of the plurality of prosthetic leaflets. Each commissure may be formed by an attachment of one of the two side edges of one of the plurality of prosthetic leaflets with one of the two side edges of an adjacent one of the plurality prosthetic leaflets.

### BRIEF DESCRIPTION OF THE DRAWINGS

FIG. 1 is a side view of a small-waisted prosthetic heart valve in an expanded, deployed condition.

FIG. 2 is a view of a cut pattern of a frame of the prosthetic heart valve of FIG. 1.

FIG. 3 is an enlarged view of the commissure attachment feature of the frame of FIGS. 1-2.

FIG. 4 is a view of a cut pattern of the commissure support structure of FIG. 1

FIG. 5 is an enlarged view of a portion of the cut pattern of FIG. 4.

FIG. 6 is a side view of a large-waisted prosthetic heart valve in an expanded condition.

FIG. 7 is a view of a cut pattern of a frame of the prosthetic heart valve of FIG. 6.

FIG. 8 is a perspective view of the frame of the prosthetic heart valve of FIG. 6, isolated from other components of the prosthetic heart valve, in an expanded condition.

FIG. 9 shows a simplified illustration of a portion of a fixture which may be used for setting the shape of the frame of FIG. 2.

FIG. 10 shows a simplified illustration of a portion of a fixture which may be used for setting the shape of the frame of FIG. 7.

FIG. 11 is a schematic representation of a leaflet of the prosthetic heart valve of FIG. 6.

FIG. 12 illustrates the prosthetic heart valve of FIG. 6 in a stage of manufacture.

FIG. 13 illustrates the inflow end of the prosthetic heart valve of FIG. 12.

FIG. 14 illustrates the outflow end of the prosthetic heart valve of FIGS. 12-13 after assembly is complete.

### DETAILED DESCRIPTION OF THE DISCLOSURE

As used herein, the term inflow, when used in connection with a prosthetic heart valve, refers to the end of the prosthetic heart valve through which blood first flows when flowing in the antegrade direction, and the term outflow refers to the end of the prosthetic heart valve through which blood last flows when flowing in the antegrade direction. Further, although the disclosure focuses on prosthetic tricuspid valve replacements, the disclosure may apply with similar force to prosthetic mitral valve replacements. Thus, unless otherwise expressly specified, the embodiments described herein may be used for replacing either a native tricuspid valve or a native mitral valve (with or without additional modifications specific to the heart valve being replaced), even if a particular embodiment may be more suited for replacing either the native tricuspid valve or the native mitral valve.

As explained in the background of the disclosure, prosthetic heart valves that include an anchoring frame and a valve frame nested within the anchoring frame typically have larger sizes when collapsed within a delivery device. As an example, this type of prosthetic heart valve may only fit within a delivery device that has a catheter with an inner diameter that is 30-33 French (10-11 mm in diameter) or larger. For transcatheter prosthetic mitral or tricuspid valves that are delivered intravascularly through the femoral vein, a delivery catheter having an outer diameter of 30-33 French or larger may increase the likelihood of access site complications, which may require a surgeon to intervene. The prosthetic heart valves disclosed herein have features and configurations that are intended to allow for the prosthetic heart valves to reliably anchor within the larger size annulus of the tricuspid valve (or the mitral valve) while being able to collapse into a delivery catheter having an inner diameter of 30-33 French or smaller. It should be understood that, as used herein, the unit French refers to the inner diameter of a catheter when describing the ability of a valve to fit within that catheter, whereas the unit French refers to the outer diameter of the catheter when describing how catheter size may result in vascular access problems.

As is described below, one way to achieve this functionality is to design the prosthetic heart valve with a single support stent (e.g., a single stent layer or a non-nested frame configuration) that can span the large atrioventricular valve annulus diameters found in patients who experience heart failure. The geometry of the support stent may allow for prosthetic leaflets to be secured inside, with atrial and/or ventricular flanges or disks that have a large enough diameter or profile to sandwich, clamp, or overlie the native annulus tissue therebetween. To provide adequate sealing between the support stent and the native valve annulus, fabric(s) may span the gap between the atrial and ventricular disks, where the fabric(s) are capable of elongating to mitigate the effects of foreshortening when sheathing the prosthetic heart valve into the catheter. It should be understood that, although the terms “frame” and “stent” are generally used interchangeably herein, the term “stent” does not imply any special structure or function beyond being a frame.

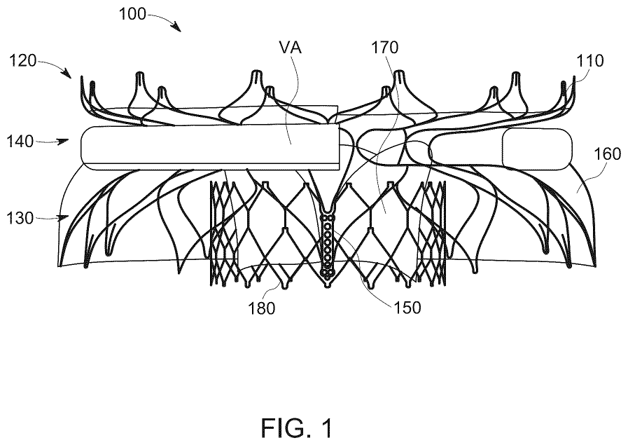

FIG. 1 is a side view of a prosthetic heart valve 100, in particular a prosthetic atrioventricular valve, shown in an expanded, implanted condition. Generally, prosthetic heart valve 100 includes a frame 110, a skirt 160, prosthetic leaflets 170, and a support ring 180. In FIG. 1, the skirt 160 is shown as partially transparent so that internal components of the prosthetic heart valve 100 are visible. Furthermore, a representation of a portion of a native heart valve annulus VA is shown in FIG. 1 with one quarter of the circumference of the valve annulus VA cut-away to better illustrate how a waist of the frame 110, described in greater detail below, engages the valve annulus VA.

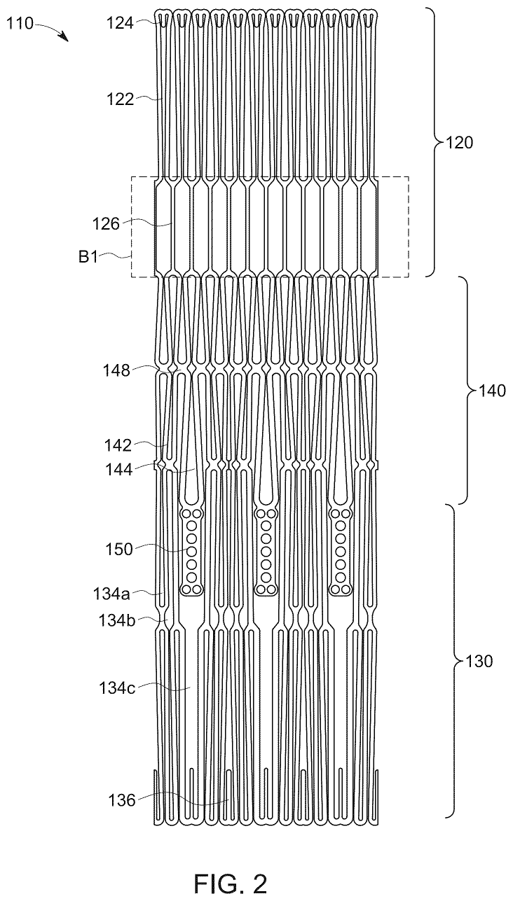

FIG. 2 shows a cut pattern of frame 110, as if the frame 110 were cut longitudinally and laid on a table. Referring to FIGS. 1 and 2, frame 110 may include an atrial flange 120 (which may alternately be referred to as an atrial disk or anchor), a ventricular flange 130 (which may alternately be referred to as a ventricular disk or anchor), and a central frame portion 140 (which may be referred to as a central waist). The frame 100 may also include a plurality of commissure attachment features (“CAFs”) 150 for use in coupling the prosthetic valve leaflets 170 to the frame 110.

Frame 110 is preferably formed of a biocompatible shape memory or superelastic material. One suitable example of this frame material is a nickel-titanium alloy, such as Nitinol. However, other materials may be suitable. In one example, frame 110 may be formed by laser cutting a hollow tube of Nitinol, and then shape-setting the frame 110 to the desired shape, for example by heat treatment. With this configuration, the frame 110 may take the set shape, such as that shown in FIG. 1, in the absence of applied forces. To deliver the prosthetic heart valve 100, the prosthetic heart valve 100 may be collapsed to a small diameter and positioned within a delivery catheter to be passed intravascularly through the patient into the patient's heart.

Referring to FIGS. 1-2, the frame 110 may be formed with a plurality of rows of generally diamond-shaped cells. In the illustrated example, the atrial flange 120 includes an inflow row of cells 122, which may include a total of twelve cells. A pin 124 may be formed at the inflow apex of one, some, or each cell 122, the pin extending a short distance in the outflow direction to a free end. Each pin 124 may be sized and shaped so that a suture loop of the delivery device may slip over the pin 124, keeping the frame 110 connected to the delivery device during delivery and deployment. Upon deployment of the prosthetic heart valve 100, each suture loop may be pushed forward or distally to disengage with the corresponding pins 124 to fully decouple the prosthetic heart valve 100 from the delivery device. Similar pins and suture loops are described in more detail in U.S. Pat. No. 10,874,512, the disclosure of which is hereby incorporated by reference herein. The atrial cells 122 may terminate, at their outflow ends, at an inflection point 148. When the frame 110 is shape-set to the desired shape, which may be generally similar to that shown in FIG. 1, the inflection points 148 may define the smallest diameter of the center portion 140. It should be understood that the term “inflection point” is not necessarily used according to its mathematical definition, but rather references the point at which the frame 110 changes from decreasing diameter to increasing diameter.

A plurality of transition cells 142, which may be generally diamond-shaped, may be positioned in a row that is adjacent to the atrial cells 122 in the outflow direction. Transition cells 142 may include an inflow portion on the inflow side of center portion 140 and an outflow portion on the outflow side of center portion 140. In some examples, the transition cells 142 may be axially centered about the inflection point 148. The row of transition cells 142 may include three enlarged transition cells 144 (or more or fewer than three depending on the number of prosthetic leaflets included in the prosthetic heart valve 100) that terminate in a commissure attachment feature (“CAF”) 150. Preferably, the enlarged transition cells 144 are positioned at substantially equal circumferential intervals around the frame 110. As best shown in FIG. 2, the sides of the atrial cells 122 (which may extend to the inflow apex of the transition cells 142 and the enlarged transition cells 144) may include elongated beams 126. These elongated beams 126 may provide additional flexibility to the atrial flange 120 (which may be referred to as the atrial disk). For example, depending on the numbers of cells included in the atrial portion, and the desired diameter that the atrial portion will span, the length of the beams 126 may be adjusted. As the desired diameter of the atrial portion 120 increases, the length (in the axial direction) of the diamond-shaped cells that form the atrial portion 120 may need to increase if a particular opening angle (e.g., about 90 degrees) of the diamond-shaped cells is desired. As the axial length of the diamond-shaped cells increases in the differently-sized valve frames, the beams 126 may correspondingly increase or decrease in length. However, in some embodiments, the beams 126 may be omitted and the atrial row of cells 122 may all be “full” diamond-shaped cells.

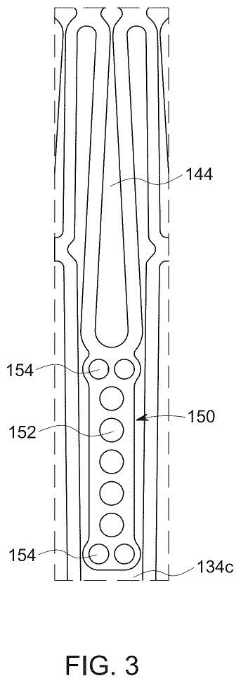

Each CAF 150 may serve as an attachment point to the prosthetic leaflets 170. For example, each CAF 150 may include a plurality of holes, and sutures may be used to couple adjacent pairs of leaflets to the CAFs 150 via the holes therein. CAFs 150 are described in greater detail below in connection with FIG. 3.

The portion of the frame 110 in the outflow direction of the inflection point 148 may include a plurality of ventricular cells. For example, a group of first ventricular cells 134a which may be generally diamond-shaped cells, the inflow apex of which is an inflection point 148. A group of second ventricular cells 134b may extend to the outflow-most portion of the frame 110, the inflow apices of the second ventricular cells being connected to the outflow apices of the transition cells 142. Some, none, or all of the second ventricular cells 134b may include tines 136, described in greater detail below, that may act as frictional engagement members that frictionally engage native tissue for enhancing securement of the frame 110 within the native valve annulus. A group of third ventricular cells 134c may be positioned between certain pairs of second ventricular cells 134b, and may include struts that extend from the inflection point 148 to the terminal outflow end of the ventricular portion 130. Third ventricular cells 134c may be larger than the other ventricular cells and may be formed in part by the struts of enlarged transition cells 144 that terminate at CAFs 150. With this configuration, at least in the cut pattern shown in FIG. 2, the CAFs 150 may be thought of as either nested within third ventricular cells 134c or forming a boundary of third ventricular cells 134c.

In addition to tines 136 being positioned in some, none, or all of the second ventricular cells 134b, none, some, or all of the third ventricular cells 134c may include tines 136. In the embodiment of FIG. 2, only some of the second ventricular cells 134b include tines 136, such that each second ventricular cell 134b that includes a tine 136 includes a single tine 136 extending upward (in the inflow direction) from an outflow apex of the cell. All of the tines 136 may extend to a free tip that may have a sharp or blunt point, that is intended either to pierce tissue or to frictionally engage the tissue without piercing it. It should be understood that the number and positioning of the tines 136 may be different from those shown in FIGS. 1-2, and the specific number and positioning shown in FIGS. 1-2 is merely exemplary. It should also be understood that, although the tines 136 are shown in FIG. 1 as being generally within the plane of the ventricular cells, in use, the tines 136 may extend radially outward (and in some embodiments through the skirt 160) from the ventricular cells to be better situated for frictionally engaging tissue.

In the illustrated embodiments, the tines 136 may be connected at an outflow end of the tine, with the free tip being positioned at an inflow end of the tine. This directionality of tines, compared to the tines being connected at their inflow end and having free tips at their outflow ends, may allow for a smoother and easier deployment of the valve from the delivery catheter. In other words, as the valve begins to self-expand as it is released from the delivery catheter, the tines do not begin to expand until the entire tine is free of the delivery device. With the opposite orientation, the tines might otherwise begin to extend radially outwardly and into contact with the end of the delivery sheath, which might make deployment more difficult. However, it should be understood that the illustrated directionality of tines may make the loading process slightly more difficult compared to the opposite directionality. However, smooth and easy deployment is typically more important than smooth and easy loading, and the loading process can be highly controlled and is performed outside the patient, while the deployment process is performed inside the patient.

After forming the frame 110 by using the cut pattern shown in FIG. 2, or another generally similar cut pattern, the frame 110 may be shape-set, for example via heat treatment, to the desired shape. FIG. 1 illustrates one example of frame 110 that has a cut pattern similar to that shown in FIG. 2, after having been shape set and having been connected to a skirt 160, prosthetic leaflets 170, and a commissure support 180, described in greater detail below.

As can be seen in FIG. 1, when the frame 110 is in the expanded or deployed condition the bottom of the atrial portion 120 may be substantially straight with a slight upward angle, with the top half of the atrial portion 120 may flare upwardly so that the tips of the atrial cells 122 point generally in the inflow direction. The contours described above may be other than exactly described while still being suitable for use in the prosthetic heart valve 100.

Still referring to FIG. 1, the ventricular portion 130 may form a general “bell” shape with a more rounded and less flat contour compared to the atrial portion 120. The more gentle contour of the ventricular portion 130 may allow for the ventricular portion 130 to drape against the ventricle and apply only light pressure to assist in fixing or otherwise securing the prosthetic heart valve 100 to the native valve annulus. This light pressure or draping may be a first mechanism by which the prosthetic heart valve 100 achieves fixation within the native valve annulus.

The various tines 136 described above may be shape set so that the free ends of the tines 136 are positioned away from the surfaces defined by the cell in which the tine 136 is located. In other words, the tines 136 may be bent or shaped so that the tips are available to pierce tissue or to frictionally engage tissue without piercing to provide a second mechanism by which the prosthetic heart valve 100 achieves fixation within the native valve annulus. The tines 136 may be oriented at different angles to achieve different objectives. For example, in some embodiments, some or all of the tines 136 may be oriented or angled with the free ends pointing toward the atrial portion 120 at an acute angle relative to the longitudinal axis passing through the center of the prosthetic heart valve 100. Tines 136 pointing at an acute angle, compared to a right angle or an obtuse angle, may be less likely to perforate tissue at the native valve annulus. Patients that may need a prosthetic atrioventricular valve, particularly a prosthetic tricuspid valve, may be likely to have very thin medial walls in the ventricle, and acutely angled tines 136 may particularly reduce the likelihood of the medial wall getting perforated by the tines 136. There may be additional benefits to having an acutely angled tine 136 compared to tines 136 with larger angles (e.g., right angle or obtuse angle), relating to loading and deployment of the prosthetic heart valve 100. For example, if the tines 136 are more acutely angled, they may provide less resistance when the prosthetic heart valve 100 is loaded into, or deployed from, the delivery catheter. Less resistance may equate to a more manageable load, which-all else being equal—may allow for a smaller size delivery catheter to be used. However, this is just one option. Some or all of the tines 136 may instead be shape set to be oriented more laterally, for example a relatively large acute angle, or a right or obtuse angle, relative to the central longitudinal axis of the prosthetic heart valve 100. Although the tines 136 may be optional entirely, if the tines 136 are included, whether they are acutely or laterally oriented, the tines 136 may provide a second mechanism by which the prosthetic heart valve 100 achieves fixation within the native valve annulus.

FIG. 3 shows an enlarged view of one of the CAFs 150. CAF 150 may be coupled to two struts at the outflow end of an enlarged transition cell 144. As noted above, CAF 150 may either be thought of as forming a boundary of, or being nested within, ventricular cells 134c. Each CAF 150 may have a general beam-shape and may include a single column of eyelets 152, with the column of eyelets 152 including a horizontal or circumferential row of two eyelets 154 flanking the opposite ends of the column of eyelets 152. As a result, the CAF 150 may have a general “dog-bone” shape. The column of eyelets 152 may provide for multiple options for a stable attachment of the commissures of the prosthetic leaflets 170 to the CAF 150, and the general shape of the CAF 150 does not foreshorten as the frame 110 expands, as may occur with diamond-shaped features. The horizontal pairs of eyelets 154 may help provide additional stability of the commissure tissue sutured through the eyelets 154 in the axial or flow direction of the prosthetic heart valve 100.

Before describing the support member 180 in more detail, an exemplary sealing skirt 160 that may be used with the prosthetic heart valve 100 is described. Referring to FIG. 1, an outer sealing skirt 160 may be provided on the exterior of the frame 110. In the particular example shown in FIG. 1, the sealing skirt 160 may be a single piece of material (although in some embodiments it may be a multi-piece design), which may be formed as a knitted fabric or a woven fabric (e.g., polyethylene terephthalate (“PET”), polytetrafluoroethylene (“PTFE”), ultra-high molecular weight polyethylene (“UHMWPE”), polyester, or similar materials). In the illustrated embodiment of FIG. 1, the sealing skirt 160 may have an atrial skirt portion and a ventricular skirt portion. The atrial skirt portion may be coupled to the atrial portion 120 of the frame 110 with a relatively tight connection—for example via suturing along the struts of the atrial portion 120 of the frame 110. In some embodiments, including that shown in FIG. 1, the inflow edge of the atrial skirt portion may be positioned a spaced distance from the atrial tips of the atrial cells 122. For example, in some embodiments the atrial end of the frame 110 inflects towards the atrium, and thus outer sealing skirt 160 may be terminated a spaced distance from the atrial end so that there is not a thrombogenic profile on the inflow end of the frame 110. In other embodiments, the inflow edge of the atrial skirt portion may be positioned to align with or cover the atrial tips of the atrial cells 122. It should be understood that the various tines 136 preferably pierce through the sealing fabric 160 so that the free ends thereof are available for frictional engagement with the native tissue upon implantation.

Still referring to FIG. 1, the ventricular skirt portion may be more loosely connected to the ventricular portion 130 of the frame 110 than the atrial skirt portion is connected to the atrial portion 120. For example, the outflow edge of the ventricular skirt portion may be relatively tightly coupled to the outflow end of the ventricular portion 130 of the frame 110, but the connection of the sealing skirt 160 may be relatively loose between the central portion 140 and the terminal end of the ventricular portion 130 of the frame 110. With this configuration, during ventricular systole (e.g., as the ventricle contracts, the prosthetic leaflets 170 close, and the pressure in the ventricle is greater than the pressure in the atrium), the pressure differential causes the ventricular skirt portion to billow, inflate, or parachute open. As the ventricular skirt portion parachutes during ventricular systole, it may fill any gaps, crevices, or openings between the prosthetic heart valve 100 and the native valve annulus that might otherwise result in blood leaking around the outside of the prosthetic heart valve 100 back into the atrium (i.e., PV leak).

Referring still to FIG. 1, the illustrated configuration of frame 110 may provide a levering effect that may further assist with scaling against PV leak. For example, when the frame 110 is in the expanded or deployed state shown in FIG. 1, deformation of the ventricular portion 130 may tend to lever the atrial portion 120 toward the ventricular portion 130. Thus, as the ventricular skirt portion inflates or parachutes during ventricular systole, which may cause the ventricular portion 130 of the frame 110 to slightly deform, the atrial portion 120 of the frame 110 may be lightly pulled downward against the atrial side of the native valve annulus. This “sandwiching” action may further seal against any PV leak, and may also mitigate potential embolization. For example, particularly in the low flow environment of the right heart, any gaps or spaces left between the prosthetic heart valve 100 and the native anatomy may create a thrombus risk zone. The above-described levering or sandwiching effect may reduce or eliminate any such gaps or spaces, thus reducing the risk of thrombus formation. In one particular example, patients may have a pronounced septal bump, and some patients may have in particular a septal bump in the right ventricle that overhands the tricuspid valve annulus. This anatomy may be an exclusion criterion for a transcatheter prosthetic tricuspid valve replacement. However, the sandwiching or levering effect described above may allow for prosthetic heart valve 100 to be implanted into patients who have relatively pronounced septal bumps.

Referring still to FIG. 1, in the deployed or expanded condition of the frame 110, the bottom struts of the enlarged transition cells 144, to which the CAFs 150 are connected, extend in the outflow direction substantially parallel to the central longitudinal axis of the prosthetic heart valve 1000. With this positioning, the CAFs 150 may be positioned in alignment with, or nearly in alignment with, the smallest diameter portion of the frame 110 at the central portion 140. In other words, the CAFs 150 of frame 110 are effectively cantilevered. This cantilevering of the CAFs 150, if no additional support is provided, may result in certain disadvantages. As explained above, the prosthetic leaflets 170 are coupled to the CAFs 150. As a result, during ventricular systole when the prosthetic leaflets 170 are closed and pressure is applied in the ventricular-to-atrial direction, the CAFs 150 and the struts of the enlarged transition cells 144 to which the CAFs 150 are attached may deflect radially inwardly toward each other. Although some amount of deflection may be desirable, the length of the CAFs 150 may be such that a risk of over-deflection may result. If the CAFs 150 deflect too much during ventricular systole, the prosthetic leaflets 170 may not coapt correctly, leading to inefficient valve functionality. Also, another disadvantage of large amounts of deflection of the CAFs 150 is that the struts from which the CAFs 150 extend may fatigue rapidly, possibly leading to failure of the frame 110.

Other potential disadvantages may result if the CAFs 150 of frame 110 do not have additional support. For example, as the prosthetic heart valve 100 is deployed from a delivery device, the ventricular or outflow end of the frame may exit the delivery device first, and begin to self-expand while the atrial end or inflow end of the frame remains collapsed within the delivery device. While the atrial portion 120 remains within the delivery device, a lever type of effect may result in which the CAFs 150 tend to splay radially outwardly as the prosthetic heart valve 100 begins to deploy from the delivery device. If the CAFs 150 are not separately supported, the CAFs 150 may tend to splay to a position that is radially outward of the shape-set position. As a result of this splaying, the prosthetic leaflets 170 may be pulled or stretched. Even if this splaying occurs temporarily during delivery, the prosthetic leaflets 170 (and/or the sutures connecting the prosthetic leaflets 170 to the CAFs 150) may be damaged, stressed, or otherwise weakened enough to cause a risk that the prosthetic leaflets 170 may either not function correctly upon implantation, or even if the prosthetic leaflets 170 function appropriately upon implantation, the longevity of the prosthetic leaflets 170 may be reduced as a result of the stress during splaying of the CAFs 150.

A third potential disadvantage may result if the CAFs 150 of frame 110 do not have additional support. Because the CAFs 150 are connected to the ventricular portion 130 of the frame 110, deformation of the ventricular portion 130 of the frame may result in deformation of the CAFs 150, and particularly their positions relative to each other. When the ventricular portion 130 deforms, the CAFs 150 may deform out of their generally circular or cylindrical alignment. This may be undesirable because as the CAFs 150 deform away from their shape-set, generally circular or cylindrical alignment, the prosthetic leaflets 170 become less likely to properly coapt with each other to form a seal.

In order to address any one or more of the potential disadvantages of CAFs 150 that exclude additional support members, a commissure support member 180 (which may be referred to herein as a CAF support or simply a support member) may be provided. The CAF support 180 is shown assembled to the frame 110 in FIG. 1. The CAF support 180 may take various forms, but in some examples it may be an expandable and collapsible ring-shaped structure.

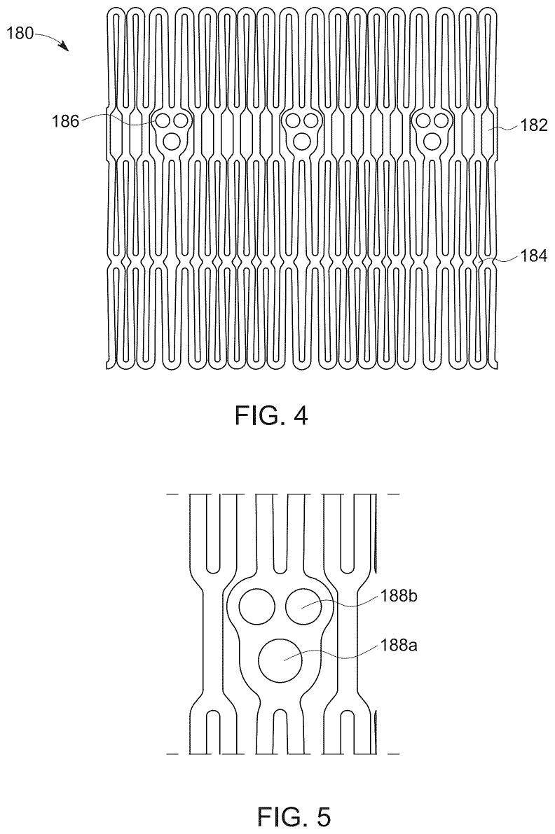

FIG. 4 shows a cut pattern for one example of CAF support 180. In the embodiment of FIG. 4, the CAF support 180 is formed of a shape-memory material, such as Nitinol, and may be laser-cut from a Nitinol tube using a pattern similar to that shown in FIG. 4. In the illustrated embodiment, commissure support 180 includes a first row of cells 182 and a second row of cells 184, and integrated connectors 186 provided on the commissure support 180. As shown in the enlarged view of FIG. 5, the connector 186 may have a partial “dog-bone” shape that includes a central eyelet 188a and two eyelets 188b arranged in a horizontal pair adjacent to the central eyelet 188a. In this particular embodiment, the three eyelets of the connector 186 may provide for a three-point connection to the corresponding CAF 150. In particular, the two horizontally arranged eyelets 188b may align with either pair of eyelets 154, with the central eyelet 188a aligning with an eyelet 152 in the column of eyelets of the CAF 150 positioned adjacent to the relevant pair of horizontal eyelets 154.

The symmetry of the eyelets in the CAF 150 may allow for the commissure support 180 to be coupled to the frame 110 in two different orientations. For example, the commissure support 180 may be coupled to frame 110 with the connectors 186 aligned with the inflow side of the CAFs 150, whereas in other embodiments, the opposite orientation may be used, in which the connectors 186 are aligned with the outflow side of the CAFs 150. In either orientation, the commissure support 180 may generally overlie the same portions of the frame 110. In other words, cither orientation of the commissure support 180 may be used relative to the frame 110 without any significant deviation in the resulting functionality, but it may be desirable to have different options for assembly.

Although not shown in FIG. 1, a buffer material may be provided between the contact points of the commissure ring 180 and the frame 110, so that there is no or minimal direct metal-to-metal contact. Any buffer material may be suitable, including fabric materials or tissue materials, and similar buffer materials may be provided with other embodiments described herein to prevent or minimize metal-to-metal contact between a frame and a commissure support.

After using the cut pattern of FIG. 4 on a tube of Nitinol (or other material), the resulting structure of commissure support 180 may be shape set (e.g., via heat treatment) so that, in the absence of applied forces, the CAF support 180 forms a generally circular or cylindrical ring. In the expanded or unbiased condition, the interior diameter of CAF support 180 is about equal to the diameter of a circle that is aligned with the outer surfaces of the CAFs 1150 when the frame 110 is in its expanded or unbiased condition.

The CAF support 180 may be positioned on the exterior of the CAFs 150 (and/or the cell struts from which the CAFs 150 extend) and coupled to the frame 110 via any suitable mechanism. For example, in some embodiments, the CAF support 180 may be simply sutured to the CAFs 150 and/or to the cell struts from which the CAFs 150 extend. Although suturing is described as one mechanism of fastening the CAF support 180 to the frame 110, it should be understood that other methods, such as adhesives, rivets (or other mechanical fasteners), etc. may be similarly suitable.

Commissure support 180 is shown and described above as being shape-set or otherwise configured into a generally circular or cylindrical shape, which would generally match the shape of the perimeter of the CAFs 150 when the prosthetic heart valve 100 is expanded and/or deployed. However, in some cases, the prosthetic leaflets 170 may open (e.g., during atrial systole) to an extent that would tend to extend radially outward of a circular perimeter formed along the CAFs 150. In other words, if the commissure support 180 was formed as a circle and coupled to the outer surfaces of the CAFs 150, the prosthetic leaflets 170 might be at risk of contacting the inner surface of the commissure support 180 when the prosthetic leaflets 170 open. This type of contact would generally be undesirable. In order to mitigate this concern, commissure support 180 may be shaped to provide clearance for the prosthetic leaflets 170 when they open, for example shaping the commissure support as a general ring structure with three enlarged lobes between each pair of connectors 186.

In use, even if the ventricular portion 130 of the frame 110 undergoes a significant amount of ovalization from forces acting on the frame 110, the commissure support 180 (and thus the CAFs 150) may maintain an almost perfect circular profile. Further, in use, the commissure support 180 may prevent the CAFs 150 from deflecting inwardly during ventricular systole more than desired, while also preventing the CAFs 150 from splaying outwardly more than desired during deployment of the prosthetic heart valve 100.

As should be understood from the disclosure provided herein, in some embodiments, a prosthetic heart valve includes a single frame (e.g., a Nitinol frame) with a commissure support member to facilitate the prosthetic heart valve having a minimal profile with a wide treatment range of annular anatomy. The frame design and/or the commissure support member help to minimize the pressure of which the prosthetic heart valve exerts against the native valve annulus. The single layer frame helps to enable the prosthetic heart valve to be compressed inside of a catheter with a small diameter (e.g., <33 French or <30 French), with scaling achieved in part by a sealing fabric. The commissure support may help to ensure the long-term durability of the prosthetic leaflets and the ventricular portion of the frame. And while the disclosure provided herein may be applied to prosthetic heart valves for replacing mitral or tricuspid valves, these features may work particularly well with the tricuspid valve due to the lower ventricular pressures involved, which may reduce the need for a bulkier two-piece frame design. In addition, the native tricuspid valve does not have the more pronounced fibrous structure found in the native mitral valve. Thus, instead of utilizing the native structure surrounding the annulus (as is often done for a prosthetic mitral valve) that works well to handle the compression other prosthetic heart valves use, the prosthetic heart valve described herein may anchor within the tricuspid valve annulus via one or more of (i) light pressure or draping of the generally bell-shaped ventricular portion of the frame; (ii) ventricular tines providing frictional engagement with the native valve annulus; and/or (iii) parachuting of the sealing skirt assisting with fixation.

It should be understood that, although the prosthetic heart valve 100 is described as including a frame 110 and a separate commissure support 180, the inclusion of the commissure support 180 does not significantly increase the profile of the prosthetic heart valve 100 when in the collapsed condition, compared to more traditional two-framed valves that may be used in mitral valve prostheses. Additional features of, and alternate embodiments of, prosthetic heart valve 100 are described in greater detail in U.S. Provisional Patent Application No. 63/384,52, filed on Nov. 21, 2022 and titled “Transcatheter Prosthetic Atrioventricular Valve with Stiffening Structure,” the disclosure of which is hereby incorporated by reference herein.

Prosthetic atrioventricular vales that are introduced via transfemoral access, including prosthetic heart valve 100, often are challenging to adapt to patients whose atrial anatomies do not have adequate height to place the device. In use, a delivery system (which contains prosthetic heart valve 100 in a collapsed condition) is used to align the collapsed valve at a certain desired position relative to the native anatomy. For example, with some self-expanding prosthetic atrioventricular valves, prior to expansion, about one-third of the length of the prosthetic valve is arranged on the atrial side of the valve annulus and about two-thirds of the length of the prosthetic valve is arranged on the ventricular side of the mitral valve annulus. This may be problematic, particularly if the length of prosthetic heart valve is long when collapsed within the delivery device, as many patients that need a prosthetic atrioventricular valve replacement may have small available space within the atrium and/or ventricle. One way to address this is by modifying the delivery device to allow for controlled deployment of the prosthetic atrioventricular valve, as described in greater detail in U.S. Provisional Patent Application No. 63/500,993, filed May 9, 2023 and titled “Ventricular Control of Prosthetic Atrioventricular Valve,” the disclosure of which is hereby incorporated by reference herein. However, another approach to address this potential problem is by decreasing the collapsed length of the prosthetic heart valve 100. The difficulty with such a change is that the prosthetic heart valve must still be able to adequately anchor within the native atrioventricular valve, adequately seal against PV leak, and adequately support the prosthetic valve leaflets so that the prosthetic valve leaflets may function appropriately.

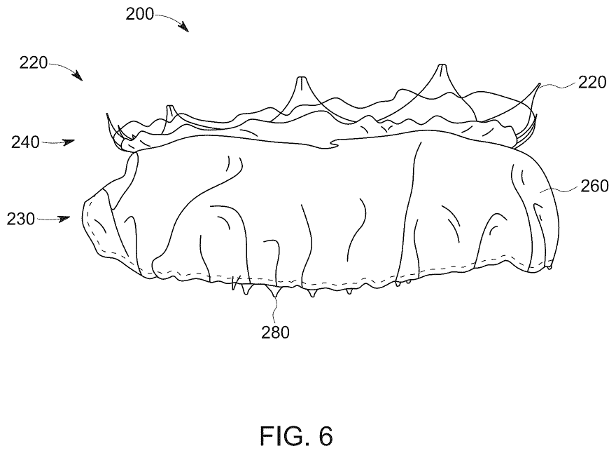

FIG. 6 illustrates a prosthetic heart valve 200 that is generally similar to prosthetic heart valve 100, but which has a significantly shorter length in the collapsed condition. Generally, prosthetic heart valve 200 may include a frame 210. Like frame 110, frame 210 may include an atrial anchor or disk 220, a ventricular anchor or disk 230, and a central or waist portion 240. Frame 210 may be similar or identical to frame 110 in most aspects, with a major differentiator being that frame 210 includes a waist portion 240 with a larger diameter, which helps results in a smaller crimped length, as described in greater detail below. Prosthetic heart valve 200 may include a commissure support 280 (only a portion being visible in FIG. 6) that is similar or identical to commissure support 180, and which is thus not described in more detail again. Prosthetic heart valve 200 may include a plurality of prosthetic leaflets 270 and a scaling skirt 260, which may both be similar or identical to prosthetic leaflets 170 and skirt 160, except for any specific differences described below.

As noted above, there are significant potential benefits to a prosthetic atrioventricular valve that has a shorter length (in the inflow-to-outflow direction) while collapsed in a delivery device. For example, such a shorter valve will be more deployable in a larger range of patients compared to longer valves. On average, the distance between a patient's tricuspid valve annulus and a position of the wall of the right ventricle aligned with the annulus is about 42 mm. However, this distance is around 20 mm for about 5% of patients. In other words, when a prosthetic atrioventricular valve is in the collapsed condition within a delivery device, it may be too long to be able to be positioned in the desired location relative to the native valve annulus before the leading/distal end of the delivery device contacts the wall of the ventricle, making it difficult or impossible to deploy the prosthetic valve in the desired configuration relative to the native valve annulus.

Prosthetic heart valve 100 may be referred to herein as the “small waist” valve design. Referring back to the small waist valve design, prosthetic heart valve 100 may have a minimum diameter of about 29 mm in the unbiased condition, which may be the diameter of the frame 110 at the point of inflection 148. This is about the same as the diameter of the valve assembly (e.g. the assembled prosthetic leaflets 170) at the inflow end. The largest expanded diameter of the atrial and/or ventricular anchors 120, 130 required to treat native atrioventricular valves (particularly the tricuspid valve) is about 75 mm. The arc length of the unbiased prosthetic heart valve frame, and resulting laser cut frame height, is generally determined by these two diameters. One way to shorten the crimped length of the prosthetic heart valve is to increase the unbiased diameter of the waist portion of the frame. For example, for each unit of distance that the unbiased diameter of the waist portion is increased, the crimped length of the prosthetic heart valve may be reduced by about 1.25 units of distance.

The “small waist” embodiment of prosthetic heart valve 100, and particularly frame 110, described in connection with FIGS. 1-2, may have an axial height or length of about 67 mm when collapsed within a delivery device. However, prosthetic heart valve 200 (which may be referred to as the “large waist” embodiment, includes frame 210, described in greater detail below, which may have a significantly smaller crimped height or length of about 53 mm (although other values, including between about 48 mm and about 58 mm may be suitable). This decrease of about 14 mm from about 67 mm (in the small-waisted embodiment) to 53 mm (in the large-waisted embodiment) may be achieved by increasing the unbiased diameter of the waist portion 240 to about 40 mm, compared to the unbiased diameter of waist portion 140 being about 29 mm (in the small-waisted embodiment). Thus by increasing the diameter of the waist portion by about 11 mm, the height of the frame may be decreased by about 14 mm (a ratio of about 1:1.25). Although this increased waist diameter of about 40 mm is one example to decrease the length of the crimped valve, it should be understood that other specific amounts of waist-diameter increase may be used for correspondingly different reductions in crimped valve length. Such shorter lengths, as described above, may allow for more patients to be treated, even those with relatively small clearance spaces available within the chamber of heart. In some embodiments, the frame 210 may come in various pre-set sizes, for example with expanded ventricular disk 230 having a diameter of about 50 mm, about 55 mm, about 60 mm, about 65 mm, about 70 mm, or about 75 mm. In some embodiments, the expanded waist diameter may be about 29 mm if the ventricular disk 230 size is about 50 mm or about 55 mm, about 32 mm if the ventricular disk 230 size is about 60 mm, about 35 mm if the ventricular disk 230 size is about 65 mm, about 40 mm if the ventricular disk 230 size is about 70 mm, or about 45 mm if the ventricular disk 230 size is about 75 mm.

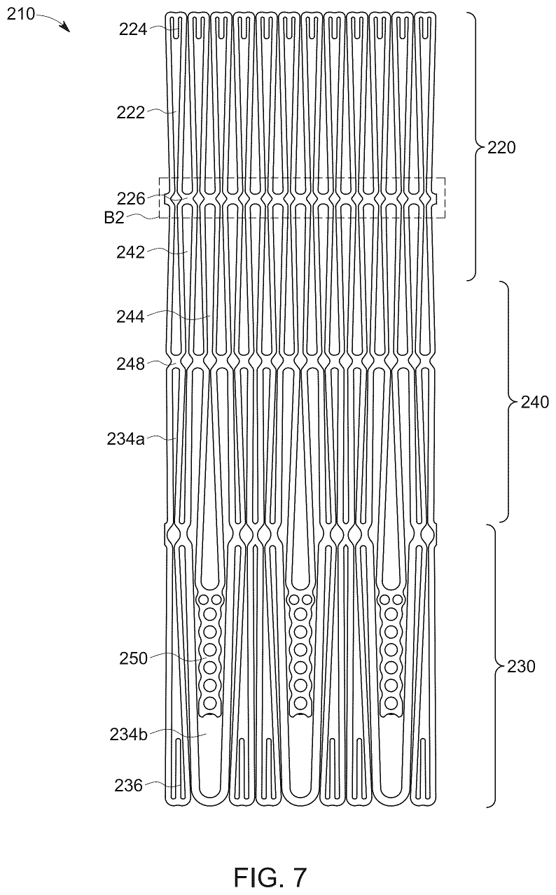

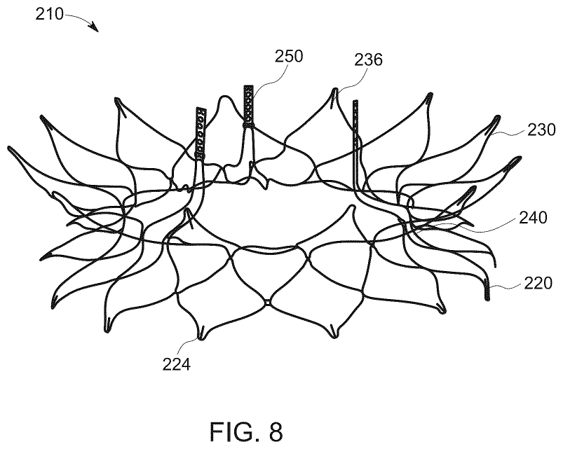

FIG. 7 illustrates a cut pattern of the frame 210 of prosthetic heart valve 200, as if cut longitudinally and laid on a table. It should be understood that the cut pattern of frame 110, shown in FIG. 2, is not provided to scale with the cut pattern of frame 210, shown in FIG. 7. FIG. 8 illustrates the frame 210, isolated from other components of the prosthetic heart valve 200, in the expanded condition. However, it should be understood that the orientation of the frame 210 in FIG. 8 is the opposite of that shown in FIG. 7. In other words, the top of the view of FIG. 7 corresponds to the inflow end, while the top of the view of FIG. 8 corresponds to the outflow end.

Referring to FIGS. 7-8, it should be understood that many features of frame 210 may be similar or identical to frame 110. For example, the materials and processes used to form the frame 210, other than the specific laser cut pattern and the specific shape to which the frames are set, may be the same, and thus are not all described in full detail again here. For example, frame 210 may include an atrial anchor or disk 220, a ventricular anchor or disk 230, and a central waist 240. Frame 210 may include CAFs 250 that are similar or identical to CAFs 150, although the struts to which the CAFs 250 connect may be slightly different compared to the corresponding struts in frame 210.

The inflow end of frame 210 may include a row of generally diamond-shaped cells 222, which may include pins 224 similar to pins 124. As with atrial cells 122, atrial cells 222 may have an outflow end that forms an inflection point 248 where the outer diameter of the waist 240 is smallest. However, one main difference between atrial cells 122 and atrial cells 222 is that, while atrial cells 112 include sides defined by elongated beams 126, the sides 226 of atrial cells 222 include no such beams (or otherwise include beams with much smaller height than beams 126). This modification leads to shortening of the atrial anchor 220, which can be readily seen by comparing the height of box B1 in FIG. 2 to the height of box B2 in FIG. 7.

As with frame 110, frame 210 may include a plurality of generally diamond-shaped transition cells 242 in a row that is adjacent to the atrial cells 222. Transition cells 242 may include an inflow portion on the inflow side of waist 240 and an outflow portion on the outflow side of waist 240. In some examples, the transition cells 242 may be axially centered about the inflection point 248. Also similar to frame 110, the row of transition cells 242 may include three enlarged transition cells 244 that terminate in CAFs 250. The structure and function of CAFs 250 may be similar or identical to CAFs 150. Although CAFs 250 are shown with only one horizontal row of apertures, CAFs 250 may be modified to be identical to CAFs 150, or vice versa.

Although CAFs 250 may be similar or identical to CAFs 150, the struts of enlarged transition cells 244 that connect to CAFs 250 may be substantially longer than the corresponding struts of enlarged transition cells 144 that connect to CAFs 150. Referring to FIG. 8, although the diameter of waist 240 has been increased (to about 40 mm in the illustrated embodiment), it is still desirable for the CAFs 250 to be positioned along a circle that is approximately 29 mm in diameter (although the specific diameter may be modified). In other words, although frames 110 and 210 have significantly different diameters at their respective waists, the positions of the CAFs 150 compared to CAFs 250 are not significantly different. For example, when the prosthetic heart valve that incorporates frame 210 is in the expanded condition, the CAFs 250 may be positioned along an imaginary circle that has a diameter which is smaller than the diameter of waist 240. In order to achieve this, instead of having CAFs 150 that extend mostly straight downwardly from inflection points 148, the struts of enlarged transition cells 244 are contoured to with two curves that allow the CAFs 250 to be positioned a distance radially inward of the points of inflection 248. In order to allow for such curvatures, the struts connecting to CAFs 250 may need to be longer than the corresponding struts connecting to CAFs 150.

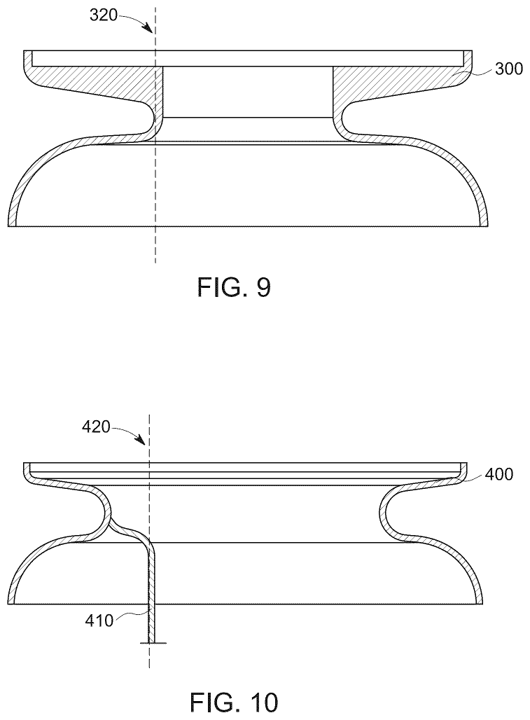

To help illustrate the differences between the shapes to which the frames 110 and 210 are set, FIG. 9 illustrates a simplified version of a portion of a fixture 300 that may be used to help set the expanded shape of frame 110, while FIG. 10 illustrates a simplified version of a portion of a fixture 400 that may be used to help set the expanded shape of frame 210. Frame 110 may be positioned around the outside of fixture 300, and clamped with another outer fixture (not shown) and heat treated so that the frame 110 has the shape of the outer contours of fixture 300 when in the unbiased or expanded condition. Referring now to FIG. 10, Frame 210 may similarly be positioned around the outside of fixture 400, and clamped with another outer fixture (not shown) and heat treated so that the frame 210 has the shape of the inner contours of fixture 400.

Comparing the fixtures 300 to 400, the waist resulting from the use of fixture 400 is significantly larger than that resulting from the use of fixture 300. Although not shown in FIGS. 9-10, the inner fixtures 300, 400 may each include slots to allow for the CAFs 150, 250 and their associated struts to pass through the interior of the inner fixtures 300, 400 for setting in a desired position. During shape setting of frame 110, the CAFs 150 and their associated struts may pass through the slots (not shown) of the inner fixture 300, and clamped using additional fixture elements (not shown) so that the CAFs 150 and their associated struts extend generally vertically or axially in general alignment with the waist of the inner fixture 300. In FIG. 9, vertical line 320 illustrates a line along which one of the CAFs 150 may be positioned during heat treatment. In other words, FIG. 9 illustrates that, after shape setting, the CAFs 150 are in alignment with the waist of the frame 110.

On the other hand, inner fixture 400 may include a commissure-setting feature 410. CAFs 250 and their associated struts may pass through slots (not shown) in the inner fixture 400 and may be pressed against the radially outer surface of the commissure-setting feature 410 during shape setting. The commissure-setting feature 410 may include a first contour extending downwardly (in the outflow direction) and inwardly from the waist area, and then a second contour extending inwardly and downwardly (in the outflow direction). With this configuration, the CAFs 250 may be positioned radially inwardly relative to the waist, while still extending substantially parallel to the longitudinal axis of the frame 210. For example, vertical line 420 illustrates a line along which one of the CAFs 250 may be positioned during heat treatment. In other words, FIG. 10 illustrates that, after shape setting, the CAFs 250 are positioned a significant spaced distance inwardly from the waist of the frame 210. As noted above, in order to help achieve this configuration, the struts connecting to the CAFs 250 may need to be longer than the corresponding struts connecting to CAF 150, to allow for such contouring to be formed. This contouring is also evident in FIG. 8.

Referring briefly again to FIG. 7, the ventricular anchor 230 of frame 210 may include a group of generally diamond-shaped first ventricular cells 234a, the inflow apex of which is an inflection point 248, and the outflow apex of which is an outflow end of the ventricular anchor 230. These cells may include tines 236 similar or identical to tines 136. The ventricular anchor 230 may include a group of second ventricular cells 234b that are generally similar to third ventricular cells 134c. The second ventricular cells 234b may be positioned between certain pairs of first ventricular cells 234a, and may include struts that extend from the inflection point 248 to the terminal outflow end of the ventricular portion 230. With this configuration, at least in the cut pattern shown in FIG. 7, the CAFs 250 may be thought of as either nested within second ventricular cells 234b or forming a boundary of second ventricular cells 234b. As should be understood from the description above, the modifications made to frame 210, compared to frame 110, allow the crimped height of the frame 210 to be significantly reduced by increasing the diameter of the waist portion 240, while still allowing the CAFs 250 to be positioned along a circle of about 29 mm. This, in turn, may allow prosthetic heart valve 200 to treat a wider selection of patients for the reasons described above.

A number of other features described in connection with prosthetic heart valve 100 are not described again in connection with prosthetic heart valve 200 because the features may be similar or identical. For example, outer skirt 260 may be substantially identical to outer skirt 160, including the sealing functionality, the outer skirt 260 being coupled (e.g., via sutures) to the waist portion 240 of the frame 210, the use and structure of the commissure support ring 280, the structure of the commissures 250 as cantilevered or floating commissures, the use of friction members such as tines 236, the use of atrial pins 234, and the way in which the atrial section of the outer skirt 260 is coupled to the frame 210.

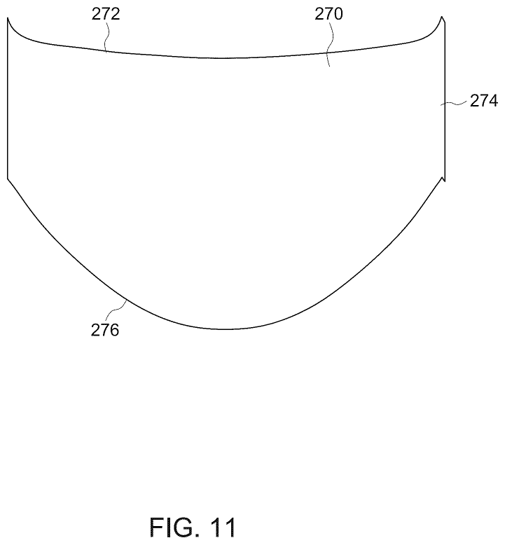

Other than changing the frame 210 compared to frame 110, the attachment of prosthetic leaflets 270 to the frame 210 may also be modified compared to prosthetic leaflets 170. For example, FIG. 11 is a highly schematic representation of one of the prosthetic leaflets 270 of prosthetic heart valve 200. As with prosthetic leaflets 170, prosthetic leaflets 270 may be formed of any suitable tissue (e.g. pericardial tissue) or synthetic material (e.g. PET, PTFE, or UHMWPE). Prosthetic leaflet 270 may include a free edge 272 at an outflow aspect of the prosthetic leaflet 270, the free edge 272 configured to move toward and away the free edges of the other prosthetic leaflets to create the valve functionality. Prosthetic leaflet 270 may include two side edges 274, with the side edge 274 of one prosthetic leaflet 270 configured to connect to a side edge 274 of an adjacent prosthetic leaflet 270, with the two side edges 274 being coupled together and to CAF 250. The prosthetic leaflet 270 may also include an attached edge 276 opposite the free edge 272 and extending between the side edges 274. The attached edge 276 may have a curved shape, including the general shape of a parabola or catenary.

It is important for the attached edges 276 of each of the prosthetic leaflets 270 to be coupled to other structures of the prosthetic heart valve 200 in order to ensure a proper seal when the free edges 272 of the prosthetic leaflets 270 are coapted with each other. Referring briefly back to FIG. 1, the attached edges of the prosthetic leaflets 170 may be sutured to seal 160 (and/or a separate fabric) at or near the waist portion 140 to help ensure that blood can only flow through the center of the assembly of prosthetic leaflets 170. This may be relatively straightforward since, for frame 110, the diameter of the waist 140 is about the same as the diameter of a circle positioned over the CAFs 150. In other words, in prosthetic heart valve 100, the attached edges of the prosthetic leaflets 170 are positioned close to other structure of the prosthetic heart valve 100 that the attached edges of the prosthetic leaflets 170 may be coupled to (e.g. via suturing). For the large-waisted prosthetic heart valve 200, on the other hand, there may be a significant gap between the attached edges 276 of the prosthetic leaflets 270 and other structures of the prosthetic heart valve 200 (other than CAFs 250) that the attached edges 276 can be coupled to. This is because, at least in part, of the larger diameter waist 240 and resulting contoured struts that attach to CAFs 250. Put simply, the attached edges 276 of the prosthetic leaflets 270 cannot simply be sutured to the waist 240 and/or sealing fabric 260 at the waist 240.

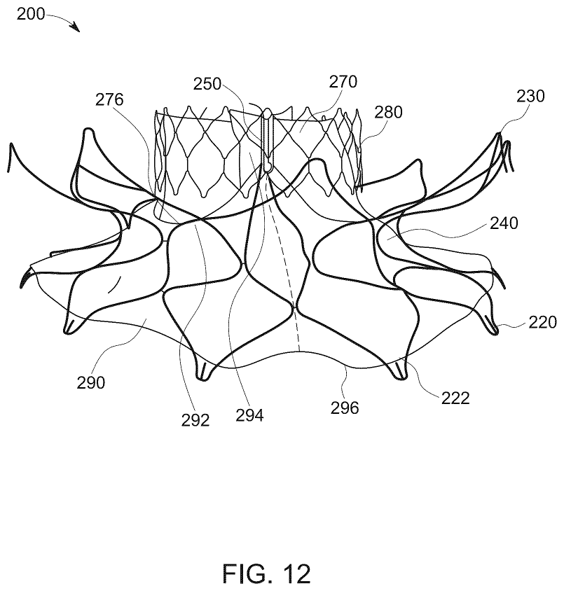

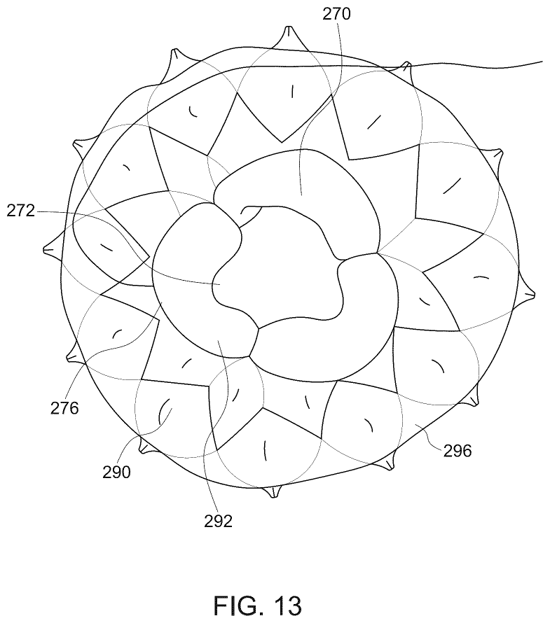

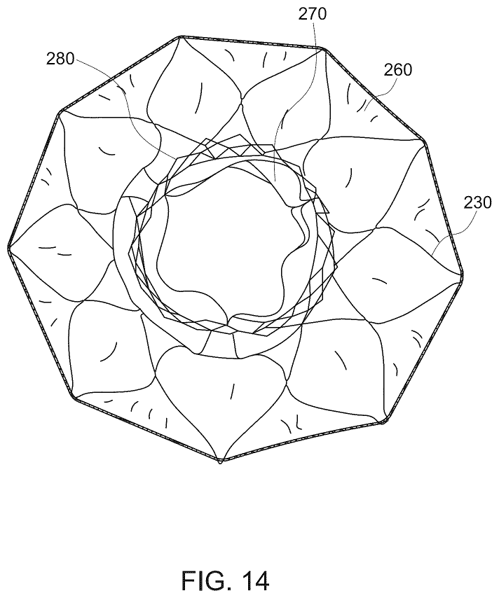

To solve the problem addressed in the paragraph above, an additional leaflet securing material may be provided. The additional leaflet securing material is preferably nonpermeable to blood to help create the desired seal with the attached edges 276. In one example, the additional material may be woven polyester, for example in the form of an interior atrial skirt 290. FIG. 12 illustrates prosthetic heart valve 200 in a stage of manufacture with prosthetic leaflets 270 mounted to the CAFs 250 within the frame 210, and the commissure support 280 mounted to the CAFs 250 on the exterior of the CAFs 250. However, in FIG. 12, the sealing skirt 260 has not yet been mounted to the exterior of the frame 210. It should be understood that, in the view of FIG. 12, the outflow end of the prosthetic heart valve 200 is positioned toward the top of the view, while the inflow end is positioned toward the bottom of the view. FIG. 13 shows the prosthetic heart valve 200 in the same stage of assembly as FIG. 12, but FIG. 13 shows the inflow end of the prosthetic heart valve 200. FIG. 14 shows the outflow end of the prosthetic heart valve 200 after assembly has been completed.

As can be seen in FIGS. 12-13, the interior atrial skirt 290 may include three contoured edges 292 that generally correspond to the shape of the attached edges 276, and each attached edge 276 may be coupled (e.g. via suturing) to the interior atrial skirt 290 along the contoured edges 292. This connection may help ensure that blood cannot flow between the attached edges 276 and the interior atrial skirt 290. In the illustrated embodiment, the interior atrial skirt 290 also includes three extensions 294 which may generally align with the CAFs 250. In one example, the extensions 294 may be positioned between the leaflet commissures formed by corresponding side edges 274 of adjacent leaflets 270 and the CAF 250 to which the commissure is attached. The interior atrial skirt 290 may include a main body portion that extends to an inflow edge 296, the inflow edge 296 being attached to the atrial cells 222. As with the sealing skirt 260, the inflow edge 296 of the interior atrial skirt 290 may stop short of the inflow-most tip of the atrial cells 222. As can be seen in FIGS. 12-13, the “tips” of the leaflets 270, which may refer to the apex of the attached edge 276, may extend beyond, and even wrap around, the inflow edge of the commissure support 280, and may even extend toward and/or wrap around the waist 240 toward the atrial anchor 220. As can be seen particularly well in FIG. 13, the atrial skirt 290 can extend toward the center of the prosthetic heart valve and the leaflet is sutured to it. These features may allow for a unique leaflet shape compared to known leaflets, for example leaflets 270 may have more of a gradual radius than the more acute radii of prior art prosthetic leaflets.

After coupling the interior atrial skirt 290, the outer or sealing skirt 260 may be coupled to the exterior of the frame 210, as shown in FIG. 14. It should be noted that the sealing skirt 260 may be coupled to the interior atrial skirt 290, for example at or near the waist 240. It should be understood that, during ventricular systole while the prosthetic leaflets 270 are closed, the CAFs 250 and commissure support 280 may provide support to the prosthetic leaflets 270. However, the pressure on the prosthetic leaflets 270 will also impart forces on the interior atrial skirt 290, particularly at and around the points of connection between the attached edges 276 to the interior atrial skirt 290. However, hydrodynamic testing has confirmed that the back pressure on the prosthetic leaflets 270 does not cause any performance problems that might be expected, such as leaflet tears, fabric tears, leakage, prolapse, or folding.

In an exemplary use of the prosthetic heart valves described herein, a prosthetic heart valve may begin in the expanded condition prior to implantation into a patient. As described above, the prosthetic heart valve may include a single monolithic or unitary stent with an outer fabric on the stent, with a commissure support member (either circular or lobed) circumscribing the commissure attachment features. The prosthetic heart valve may be drawn or otherwise forced into a delivery catheter, the prosthetic heart valve transitioning into the collapsed condition as it moves into the delivery catheter. Preferably, the outer diameter of the catheter of the delivery device has a size of 30 French (10 mm) or smaller, including 28 French (9.33 mm) or smaller or 24 French (8 mm) or smaller. With the prosthetic heart valve successfully collapsed in the small diameter delivery device catheter, the delivery device may be introduced into the patient, for example through the femoral vein, and navigated to the target site, for example the native tricuspid valve. Upon reaching the target site, the prosthetic heart valve may be deployed from the delivery device catheter, for example by retracting the delivery device catheter relative to the prosthetic heart valve. As the constraint on the prosthetic heart valve is removed, the prosthetic heart valve will naturally begin to expand as the frame tends to return to its preset shape. Preferably, the ventricular disk of the prosthetic heart valve is released first within the ventricle (e.g., the right ventricle). As the ventricular disk expands, the ventricular disk may begin to apply light pressure on the tissue, and the ventricular tines may frictionally engage (with or without piercing) the native tissue. As deployment continues, the center portion of the stent of the prosthetic heart valve will generally align with the valve annulus. As the atrial side of the prosthetic heart valve deploys, the atrial disk of the stent will expand on the atrial side of the native valve. Thus, as described above, a small delivery device may be used, despite the requirement of coverage of a large native valve annulus area, and without losing any sealing capabilities despite using only a single stent with a small center portion housing the prosthetic leaflets.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A prosthetic heart valve for replacing a native atrioventricular valve, the prosthetic heart valve comprising:
a collapsible and expandable frame, the frame including an atrial anchor, a ventricular anchor, and a center waist extending between the atrial anchor and the ventricular anchor, the frame including a plurality of commissure attachment features (“CAFs”) that include struts that extend from the center waist;
a plurality of prosthetic leaflets mounted to the plurality of CAFs; and
a sealing fabric coupled to an outer surface of the frame;
wherein in an expanded condition of the prosthetic heart valve, the atrial anchor and the ventricular anchor each flare outwardly from the center waist, the center waist defining a waist diameter of the frame,
wherein in the expanded condition of the prosthetic heart valve, each of the plurality of CAFs is spaced from adjacent ones of the plurality of CAFs so that gaps in the frame are present between adjacent ones of the plurality of CAFs; and
wherein in the expanded condition of the prosthetic heart valve, the CAFs are positioned along an imaginary circle having a diameter that is smaller than the waist diameter.

2. The prosthetic heart valve of claim 1, wherein in the expanded condition of the prosthetic heart valve, the waist diameter is about 40 mm.

3. The prosthetic heart valve of claim 2, wherein in a collapsed condition of the prosthetic heart valve, the prosthetic heart valve has an axial height between an inflow end of the prosthetic heart valve and an outflow end of the prosthetic heart valve, the axial height being between about 48 mm and about 58 mm.

4. The prosthetic heart valve of claim 1, further comprising:
a commissure support ring coupled to and extending around the plurality of CAFs.

5. The prosthetic heart valve of claim 4, wherein the commissure support ring is a collapsible and expandable structure that has a circular or lobed shape in an expanded condition of the commissure support ring.

6. The prosthetic heart valve of claim 4, wherein the commissure support ring includes a first circumferential row of generally diamond-shaped cells.

7. The prosthetic heart valve of claim 6, wherein the commissure support ring includes a second circumferential row of generally diamond-shaped cells adjacent the first circumferential row.

8. The prosthetic heart valve of claim 7, wherein the commissure support ring includes a plurality of connectors integrally formed with the commissure support ring, the plurality of connectors each having a shape that is complementary to a shape of each of the plurality of CAFs.

9. The prosthetic heart valve of claim 1, wherein the frame includes a plurality of tines on the ventricular anchor, each of the plurality of tines extending to a free end pointing toward the atrial anchor in a collapsed condition of the prosthetic heart valve.

10. The prosthetic heart valve of claim 1, wherein in the expanded condition of the prosthetic heart valve, the ventricular anchor of the frame is bell-shaped.

11. The prosthetic heart valve of claim 10, wherein the sealing fabric extends over the ventricular anchor and over the center waist of the frame, an inflow edge of the sealing fabric being positioned a spaced distance from a terminal end of the atrial anchor.

12. The prosthetic heart valve of claim 1, wherein, in the expanded condition of the prosthetic heart valve, the struts that couple the plurality of CAFs to the center waist include a first contour extending in an outflow and radially inward direction, and a second contour extending in an outflow and radially outward direction, the plurality of CAFs being coupled to the second contour of respective ones of the struts.

13. The prosthetic heart valve of claim 12, wherein, in the expanded condition of the prosthetic heart valve, the plurality of CAFs each extend in a direction substantially parallel to a center longitudinal axis of the prosthetic heart valve.

14. The prosthetic heart valve of claim 13, wherein in the expanded condition of the prosthetic heart valve, the diameter of the imaginary circle is about 29 mm.

15. The prosthetic heart valve of claim 1, wherein each of the plurality of prosthetic leaflets includes an attached edge, a free edge opposite the attached edge, and two side edges extending between the free edge and the attached edge.

16. The prosthetic heart valve of claim 15, further comprising:
an atrial skirt attached to an interior surface of the frame, the atrial skirt having an inflow edge attached to the atrial anchor, and a plurality of contoured sections, each of the plurality of contoured sections attached to a respective one of the attached edge of the plurality of prosthetic leaflets.

17. The prosthetic heart valve of claim 16, wherein the atrial skirt is formed of a material that is non-permeable to blood.

18. The prosthetic heart valve of claim 17, wherein the material is a woven polyester.

19. The prosthetic heart valve of claim 17, wherein the atrial skirt is coupled to the sealing skirt.

20. The prosthetic heart valve of claim 17, wherein the atrial skirt includes a plurality of extensions, each of the plurality of extensions being positioned between a respective one of the plurality of CAFs and a respective commissure of the plurality of prosthetic leaflets, each commissure being formed by an attachment of one of the two side edges of one of the plurality of prosthetic leaflets with one of the two side edges of an adjacent one of the plurality prosthetic leaflets.
